(19) United States
(12) Patent Application Publication
(10) Pub. No.: US 2008/0311038 A1
(43) Pub. Date: Dec. 18, 2008

(54) Radicicol Derivatives Useful for Position Emission Tomography

(76) Inventors: Yves Auberson, Allschwil (CH); Mats Bergstroem, Basel (CH); Emmanuelle Briard, Huningue (FR); Patrick Chene, Mulhouse (FR); Laurent Martarello, Chiswick (GB); Joseph Schoepfer, Riehen (CH)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/159,744

(22) PCT Filed: Jan. 2, 2007
(86) PCT No.: PCT/EP2007/000013

§ 371 Date: Jun. 30, 2008

(30) Foreign Application Priority Data
Dec. 30, 2005 (GB) 0526615.0

(51) Int. Cl. A61K 51/04; C07D 313/00

(52) U.S. Cl. 424/1.89; 549/355; 424/1.81

(57) ABSTRACT

The present invention relates to novel radicicol derivatives of the formula I,

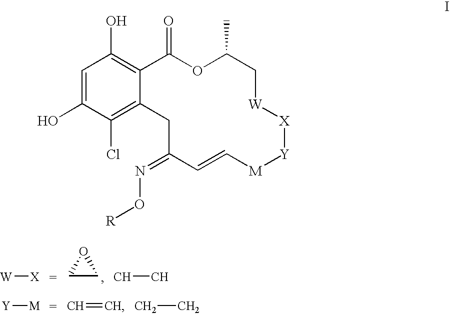

in which
R is R′, (CH₂)ₙCOOR′ or (CH₂)ₙCONHR′, wherein
R′ is ¹¹CH₃, [³H]₃C, [³H]₂HC, [³H]H₂C or (CH₂)ₙHal, wherein
Hal is ¹²³I, ¹²⁵I, ¹³¹I, I, ⁷⁵Br, ⁷⁶Br, ⁷⁷Br, ⁸²Br, Br, ¹⁸F or F, or
R′ is (CH₂)ₙ₋₁[³H]HCHal or (CH₂)ₙ₋₁[³H]₂CHal, wherein
Hal is I, Br or F, and
n is, each independently, 1, 2, 3 or 4,
in free form or in salt form, to their preparation, to their use as radiotracers/markers and to compositions containing them.

The present invention relates to novel radicicol derivatives, to their preparation, to their use as radiotracers/markers and to compositions containing them.

The Hsp90 family of chaperones is comprised of four known members: Hsp90α and Hsp90β both in the cytosol, grp94 in the endoplasmic reticulum and trap-1 in the mitochondria. Hsp90 is an abundant cellular chaperone required for the ATP-dependent refolding of denatured or “unfolded” proteins and for the conformational maturation of a variety of key proteins involved in the growth response of the cell to extracellular factors. These proteins, which are called client proteins, include the steroid receptors as well as various protein kinases. Hsp90 is essential for eukaryotic cell survival and is overexpressed in many tumors. Cancer cells seem to be sensitive to transient inhibition of Hsp90 ATPase activity suggesting that Hsp90 inhibitors could have a potential as new anticancer drugs. Each Hsp90 family member possesses a conserved ATP-binding site at its N-terminal domain, which is found in few other ATP-binding proteins. The weak ATPase activity of Hsp90 is stimulated upon its interaction with various co-chaperone proteins. Several natural compounds such as geldanamycin or radicicol bind at the ATP-binding site of Hsp90 inhibiting its ATPase activity. In cellular systems and in vivo, these drugs upon binding to Hsp90 prevent the folding of the client proteins, which are then degraded in the proteasome. 17-Allylamino-17-demethoxy-geldanamycin (17-AAG), a geldanamycin derivative, is currently in phase I clinical trials at several institutions. Initial clinical experiences with 17-AAG have offered preliminary evidence, that concentrations of the drug associated with activity in pre-clinical systems can be achieved in humans with tolerable toxicity, and provided early evidence of target modulation in at least certain surrogate and tumor compartments. The dose limiting toxicity of 17-AAG is hepatic. The poor solubility of 17-AAG makes it difficult to formulate/administer, and its synthesis is difficult (it is generally obtained by fermentation). Therefore, alternative compounds with better physicochemical properties and perhaps higher specificity (17-AAG inhibits all of the four Hsp9G paralogs) are desired.

Radicicol is a macrocyclic antibiotic shown to reverse the malignant phenotype of v-Src and v-HA-RAs transformed fibroblasts. It was shown to degrade a number of signalling proteins as a consequence of HSP90 inhibition. X-ray crystallographic data confirmed, that radicicol also binds to the N-terminal domain of HSP90 and inhibits the intrinsic ATPase activity. However, radicicol lacks antitumor activity in vivo due to the unstable chemical nature of the compound. It was found, that converting the ketone function to an oxime function affords products retaining the activity in vivo. These results have raised the possibility, that radicicol can be modified to yield agents with improved in vivo properties.

Noninvasive nuclear imaging techniques can be used to obtain basic and diagnostic information about the physiology and biochemistry of living subjects, including experimental animals, patients and volunteers. These techniques rely on the use of imaging instruments, that can detect radiation emitted from radiotracers administered to living subjects. The information obtained can be reconstructed to provide planar and tomographic images, which reveal the distribution and/or concentration of the radiotracer as a function of time.

Positron emission tomography (PET) is the noninvasive imaging technique, that offers the highest spatial and temporal resolution of all nuclear medicine imaging modalities and has the additional advantage, that it can allow for the true quantification of tracer concentrations in tissues. The technique involves the use of radiotracers labelled with positron emitting radionuclides, that are designed to have in vivo properties, which permit the measurement of parameters regarding the physiology or biochemistry of a variety of processes in living tissue.

Compounds can be labelled with positron or gamma emitting radionuclides. The most commonly used positron emitting radionuclides are ¹⁵O, ¹³N, ¹¹C and ¹⁸F, which are accelerator produced and have half lives of 2, 10, 20 and 110 minutes, respectively. The most widely used gamma emitting radionuclides are ^{99m}Tc, ²⁰¹Tl and ¹²³I.

We have now found, that certain radicicol derivatives can be used to probe Hsp90 in vitro and in vivo using molecular imaging modalities, such as PET.

Accordingly, in a first aspect, the present invention relates to a compound of the formula I,

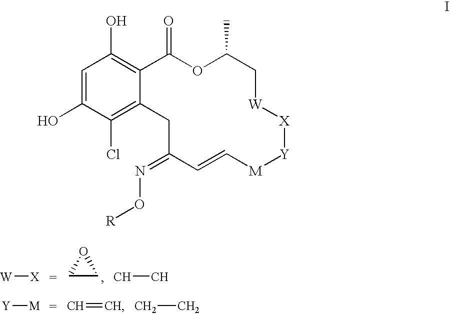

in which
R is R′, (CH₂)ₙCOOR′ or (CH₂)ₙCONHR′, wherein
R′ is ¹¹CH₃, [³H]₃C, [³H]₂HC, [³H]H₂C or (CH₂)ₙHal, wherein

### Hal is ¹²³I, ¹²⁵I, ¹³¹I, I, ⁷⁵Br, ⁷⁶Br, ⁷⁷Br, ⁸²Br, Br, ¹⁸F or F, or

R′ is (CH₂)ₙ₋₁[³H]HCHal or (CH₂)ₙ₋₁[³H]₂CHal, wherein

### Hal is I, Br or F, and

n is, each independently, 1, 2, 3 or 4,
in free form or in salt form.

In preferred embodiments, the invention relates to a compound of the formula I, in free form or in salt form, in which

(1) R is R′, (CH₂)ₙCOOR′ or (CH₂)ₙCONHR′, wherein
R′ is ¹¹CH₃, [³H]₃C, [³H]₂HC, [³H]H₂C or (CH₂)ₙHal, wherein

### Hal is ¹²³I, ¹²⁵I, ¹³¹I, ⁷⁵Br, ⁷⁶Br, ⁷⁷Br, ⁸²Br or ¹⁸F, or

R′ is (CH₂)ₙ₋₁[³H]HCHal or (CH₂)ₙ₋₁[³H]₂CHal, wherein

### Hal is I, Br or F, and

n is, each independently, 1, 2, 3 or 4;
(2) R is R′, (CH₂)ₙCOOR′ or (CH₂)ₙCONHR′, wherein
R′ is ¹¹CH₃, [³H]₃C or (CH₂)ₙHal, wherein

### Hal is ¹²³I, ⁷⁶Br or ¹⁸F, or

R′ is (CH₂)ₙ₋₁[³H]₂CHal, wherein

### Hal is I, Br or F, and

n is, each independently, 1, 2, 3 or 4.

In especially preferred embodiments, the invention relates to one or more than one of the compounds of the formula I mentioned in the Examples hereinafter, in free form or in salt form.

If asymmetrical carbon atoms are present in the compounds of the formula I, the compounds may exist in pure optically active form or in the form of mixtures of optical isomers, e.g. in the form of racemic mixtures. All pure optical isomers and all their mixtures, including the racemic mixtures, are part of the present invention.

In the case of possible stereoisomerism, e.g. cis/trans-isomerism of a double bond, the compounds may exist as pure stereoisomers or mixtures thereof. All such pure stereoisomers and all such mixtures are part of the present invention.

In a further aspect, the invention relates to a process for the preparation of a compound of the formula I, in free form or in salt form, comprising the steps of

a) for the preparation of a compound of the formula Ia,

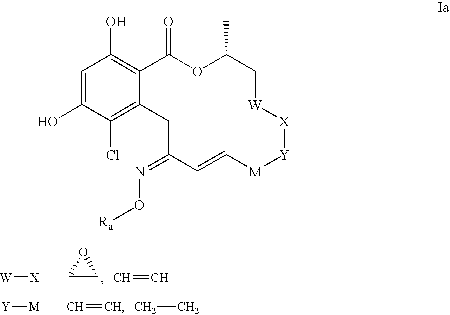

in which Rₐ is ¹¹CH₃, [³H]H₂C, [³H]₂HC or [³H₃]C, reacting a compound of the formula II

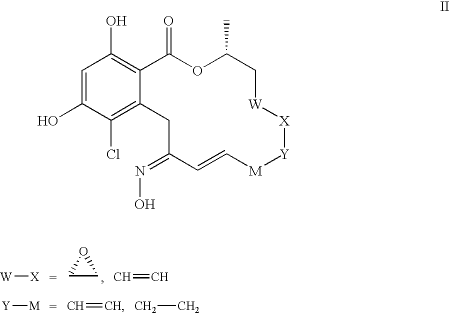

with ¹¹CH₃L, [³H]H₂CL, [³H]₂HCL or [³H]₃CL, wherein L is I, OTs, OMs or OTf, preferably in the presence of a base, or
b) for the preparation of a compound of the formula Ib,

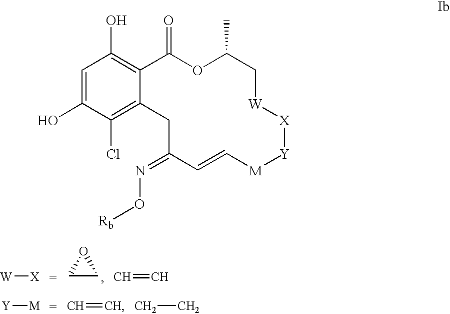

in which R_{b} is [¹⁸F](CH₂)ₙ, F(CH₂)ₙ, [¹²³I](CH₂)ₙ, [¹²⁵I](CH₂)ₙ, [¹³¹I](CH₂)ₙ, I(CH₂)ₙ, [⁷⁵Br](CH₂)ₙ, [⁷⁶Br](CH₂)ₙ, [⁷⁷Br](CH₂)ₙ, [⁸²Br](CH₂), or Br(CH₂)ₙ, reacting a compound of the formula III,

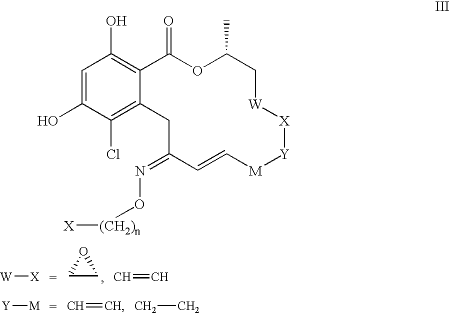

in which n is 1, 2, 3 or 4 and X [in the group ═N—O—(CH₂)ₙ—X] is OTs, OMs, OTf or 1, with [¹²³I]⁻, [¹²⁵I]⁻, [¹³¹I]⁻, I⁻, [⁷⁵Br]⁻, [⁷⁶Br]⁻, [⁷⁷Br]⁻, [⁸² Br]⁻, Br⁻, [¹⁸F]⁻ or F⁻ or reacting a compound of the formula II with a compound of the formula IV,

X—R_{b} IV

wherein X and R_{b} are as defined above in this variant, or reacting a radicicol derivative, which carries an oxo group instead of the oxime function present in the compound of the formula I, with [¹⁸F](CH₂)ₙ—O—NH₂, [¹²³I](CH₂)ₙ—O—NH₂, [¹²⁵ I](CH₂)ₙ—O—NH₂, [¹³¹I](CH₂)ₙ—O—NH₂, [⁷⁵Br](CH₂)ₙ—O—NH₂, [⁷⁶Br](CH₂)ₙ—O—NH₂, [77Br](CH₂)ₙ—O—NH₂ or [⁸²Br](CH₂)ₙ—O—NH₂, n being in each case 1, 2, 3 or 4, preferably 2, 3 or 4, or
c) for the preparation of a compound of the formula Ic,

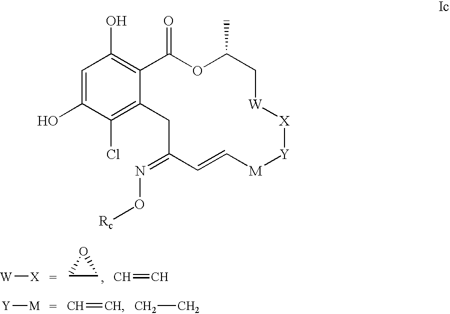

wherein R_{c} is (CH₂)ₙCO₂[¹¹C]H₃, (CH₂)ₙCO₂[³H]₃C, (CH₂)ₙCO₂[³H]₂HC or (CH₂)ₙCO₂[³H]H₂C, reacting a compound of the formula V,

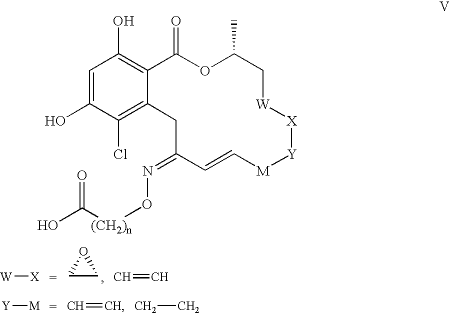

in which n is 1, 2, 3 or 4, with [¹¹C]H₃L, [³H]H₂CL, [³H]₂HCL or [³H]₃CL, wherein L is I, OTs, OMs or OTf, preferably in the presence of a base, or
d) for the preparation of a compound of the formula Id,

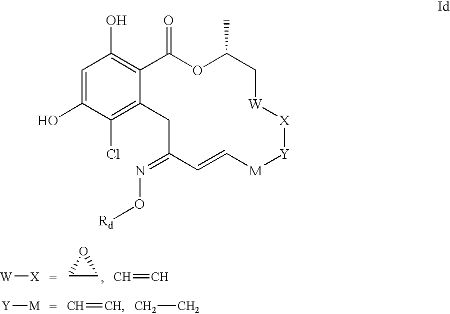

wherein R_{d} is [¹²³I](CH₂)ₙCONH(CH₂)ₙI, [¹²⁵I](CH₂)ₙCONH(CH₂)ₙI, [¹³¹I](CH₂)ₙCONH(CH₂)ₙI, (CH₂)ₙCONH(CH₂)ₙI, [⁷⁵Br](CH₂)ₙCONH(CH₂)ₙBr, [⁷⁶Br](CH₂)ₙCONH(CH₂)ₙBr, [⁷⁷Br](CH₂)ₙCONH(CH₂)ₙBr, [⁸²Br](CH₂)ₙCONH(CH₂)ₙBr, (CH₂)ₙCONH(CH₂)ₙBr, [¹⁸F](CH₂)ₙCONH(CH₂)ₙF, or (CH₂)ₙCONH(CH₂)ₙF, n being in each case, independently, 1, 2, 3 or 4, reacting a compound of the formula VI,

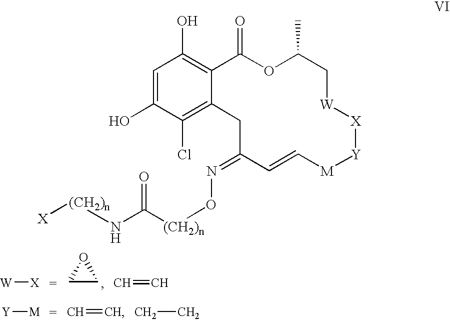

wherein n is in each case, independently, 1, 2, 3 or 4 and the terminal X is OTs, OMs, OTf or I, with [¹²³I]⁻, [¹²⁵I]⁻, [¹³¹I]⁻, I⁻, [⁷⁵Br]⁻, [⁷⁶ Br]⁻, [⁷⁷Br]⁻, [⁸²Br]⁻, Br⁻, [¹⁸F]⁻ or F⁻
or reacting a compound of the formula V, in which n is 1, 2, 3 or 4, with [¹⁸F](CH₂)ₙNH₂, F(CH₂)ₙNH₂, [¹²³I](CH₂)ₙNH₂, [¹²⁵I](CH₂)ₙNH₂, [¹³¹I](CH₂)ₙNH₂, I(CH₂)ₙNH₂, [⁷⁵Br](CH₂)ₙNH₂, [⁷⁶Br](CH₂)ₙNH₂, [⁷⁷Br](CH₂)ₙNH₂, [⁸²Br](CH₂)ₙNH₂, Br(CH₂)ₙNH₂, F[³H]₃C(CH₂)ₙ₋₁, —NH₂, F[³H]₂HC(CH₂)ₙ₋₁. NH₂, F[³H]H₂C(CH₂)ₙ₋₁NH₂, Br[³]H₃C(CH₂)ₙ₋₁NH₂, Br[³H]₂HC(CH₂)ₙ₋₁NH₂, Br[³H]H₂C(CH₂)ₙ₋₁NH₂, I[³H]₃C(CH₂)ₙ₋₁NH₂, I[³H]₂HC(CH₂)ₙ₋₁NH₂ or I[³H]H₂C(CH₂)ₙ₋₁, NH₂, n being in each case 2, 3 or 4, preferably using standard carboxylic activation methods, e.g. the formation of an acyl halide, or peptide coupling reagents,
in each case optionally followed by cleavage of protecting groups optionally present, and in each case of recovering the so obtainable compound of the formula I in free, preferably free acid, form or in the form of a, preferably phenolic, salt.

The reactions can be effected according to conventional methods, for example as described in the Examples.

The working-up of the reaction mixtures and the purification of the compounds thus obtainable may be carried out in accordance with known procedures.

Salts may be prepared from the free compounds in known manner, and vice-versa.

Compounds of the formula I can also be prepared by further conventional processes, which processes are further aspects of the invention, e.g. as described in the Examples.

The starting materials of the formulae Ia, Ib, Ic, Id, II, III, IV, V and VI are known or may be prepared according to conventional procedures starting from known compounds, for example as described in the Examples.

During any of the described synthetic sequences it may be necessary and/or desirable to protect sensitive or reactive groups on any molecules concerned. This may be achieved by means of conventional protecting groups, such as those described in Protective Groups in Organic Chemistry, ed J. F. W. McOmie, Plenum Press, 1973; and T. W. Greene and P. G. M. Wuts, Protective Groups in Organic Synthesis, Jon Wiley & Sons, 1991. The protecting groups may be removed at a convenient stage using methods known from the art. Where hydroxyl groups require protection, this may be achieved by forming, esters, trialkylsilyl, tetrahydropyran, benzyl or alkyl ethers. Such derivatives may be deprotected by standard procedures, thus, for example, a methoxymethyl ether derivative may be deprotected using hydrochloric acid in methanol.

Compounds of the formula I, hereinafter referred to as “agents of the invention”, exhibit valuable properties as histopathological in vitro and in vivo labeling agents, imaging agents and/or biomarkers, hereinafter “markers”, for the selective labeling of heat shock protein 90 (Hsp90) and as ligands targeting HSP90 (for non-radiolabelled compounds) for the treatment of diseases that HSP90 modulators can target.

Suitable radionuclides, that may be incorporated in compounds of the formula I include: ³H, ¹⁸F, ¹²³I, ¹²⁵I, ¹³¹I, ⁷⁵Br, ⁷⁶Br, ⁷⁷Br and ⁸²Br. The choice of radionuclide to be incorporated into compounds of the formula I will depend on the specific analytical or pharmaceutical application. Therefore, for in vitro labelling of HSP90 and for competition assays compounds that incorporate ³H, ¹²⁵I or ⁷⁷Br would be preferred. For diagnostic and investigative imaging agents, compounds that incorporate a radionuclide selected from ¹¹C, ¹⁸F, ¹²³I or ⁷⁶Br are preferred. Incorporation of a chelating radionuclide may be useful in certain applications.

Radiolabelled analogues of compounds of the formula I may be used in clinical studies to evaluate the role of HSP90 ligands in a variety of disease areas where HSP90 ligands are believed to be involved.

The present invention also provides a radiopharmaceutical composition, which comprises a compound of the formula I and a pharmaceutically acceptable carrier or excipient.

More particularly the agents of the invention are useful as markers for labeling Hsp90 in vitro or in vivo (see Examples 7-9).

The agents of the invention are therefore useful, for instance, for determining the levels of receptor occupancy of a drug acting at Hsp90, or for diagnostic purposes for diseases resulting from an overexpression, activation or dysregulation of Hsp90, and for monitoring the effectiveness of pharmacotherapies of such diseases.

In accordance with the above, the present invention provides an agent of the invention for use as a marker for cancer imaging or neuroimaging.

In a further aspect, the present invention provides a composition for labeling tumors, brain and other tissues involving overexpression, activation, or dysregulation of Hsp90 in vivo and in vitro comprising an agent of the invention.

In still a further aspect, the present invention provides a method for labeling tumors, brain and other tissues involving overexpression, activation, or dysregulation of Hsp90 in vitro or in vivo, which comprises contacting the tumor, brain tissue or other tissues with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

The following Examples illustrate the invention, but do not limit it (Me=CH₃).

### EXAMPLE 1

### Preparation of [¹¹C—O-methyl] radicicol 9-(O-methyl-oxime), [¹¹C]Ia (Ra=[¹¹C]CH₃)

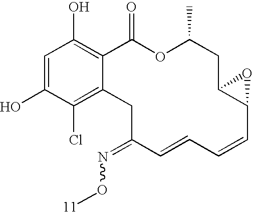

The title compound is prepared by reacting [¹¹C]MeI with the sodium salt of the oxime II in dry DMF (400 μl). [¹¹C]MeI is trapped at room into a 1 ml glass container loaded with reactants, when addition is completed the reaction mixture is heated to 120° C. for 10 min. The title compound is purified by reverse phase semi-prep HPLC. The product fraction collected are evaporated and reformulated to afford [¹¹C—O-Methyl] radicicol 9-(O-methyl-oxime).

Note: The preparation of I (from Radicicol) is known and described in Bioorg Med Chem, 2002, 3445.

### EXAMPLE 2

### Preparation of [O-tri(³H)-methyl] radicicol 9-(O-methyl-oxime), [³H]Ia (Ra=[³H]₃C)

The title compound can be prepared by reacting [³H]MeI with the sodium or potassium salt of the oxime II in dry DMF, followed by reverse phase semi-prep HPLC purification.

### EXAMPLES 3^{a}, 3b AND 3c

### Radicicol 9-(O-(2-fluoro-ethyl-oxime)

By reaction of radicicol with O-(2-fluoro-ethyl)-hydroxylamine (example 3a).

By reaction of radicicol 9-oxime with 1-[¹⁸F]fluoro-2-tosyloxyethane (example 3b).

By reaction of radicicol with [¹⁸F]O-(2-fluoro-ethyl)-hydroxylamine. [¹⁸F]O-(2-fluoro-ethyl)-hydroxylamine is prepared by alkylation of N-hydroxy-phthalimide with [¹⁸F]1-bromo-2-fluoro-ethane followed by reaction of the [¹⁸F]2-(2-fluoro-ethoxy)-isoindole-1,3-dione with hydrazine hydrate (example 3c).

### EXAMPLE 3a

### Preparation of [¹⁸F]radicicol 9-(O-fluoroethyl-oxime), Ib (Ra=C₂H₄F)

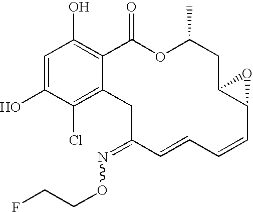

The title compound can be prepared according to literature procedure [Bioorganic & Medicinal Chemistry 2002, 19, 3445-3454], using O-(2-fluoro-ethyl)-hydroxylamine hydrochloride [The Journal of Antibiotics 2000, 53, 1071-1085]. Diastereoisomeric mixture of (9Z,11E)-(4R,6R,8R)-16-Chloro-17,19-dihydroxy-4-methyl-3,7-dioxa-tricyclo[13.4.0.0*6,8*]nonadeca-1 (19),9,11,15,17-pentaene-2,13-dione 13-[O-(2-fluoro-ethyl)-oxime], HPLC t_{R}: 5.0, (M+H)⁺=426.

### EXAMPLE 3b

### Preparation of [¹⁸F]radicicol 9-(O-fluoroethyl-oxime), [¹⁸F]Ib (Ra=[¹⁸F]C₂H₄) from II

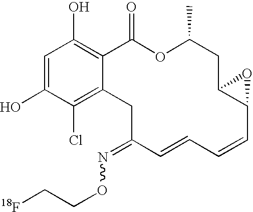

The first step consists of the preparation of 1-[¹⁸F]fluoro-2-tosyloxyethane. [18F]fluoride in solution in enriched [¹⁸O]H₂O is added to a Wheaton 5 mL micro-vial (Millville, N.J. USA) containing 1 mL of a solution of 10 mg Kryptofix (K-222) (Aldrich Chemical, Milwaukee, Wis. USA) and 1 mg potassium carbonate in 0.05 mL water and 0.95 mL CH₃CN. The solution is heated at 116° C. for 3.5 min after which three additional portions of 1 mL CH₃CN are added and evaporated to dry the fluoride. The vial is cooled to room temperature and 1.5 mg of 1,2-ditosyloxyethane dissolved in 1.0 mL CH₃CN is added. The solution was heated to 80° C. for 10 min, cooled to room temperature, diluted with 5 mL of ether, and passed through a Waters classic SiO₂ Sep-Pak (Milford, Mass. USA) into a 10 mL maxi-vial attached to a 50 ml round bottomed flask. The sep-pak was rinsed with 5 mL of Et₂O, which was added to the maxi-vial, bringing the total volume to 10 mL. The resulting ethereal solution was evaporated in vacuo and transferred to a 5 mL vial to yield [¹⁸F]fluoro-2-tosyloxyethane. The sodium salt of the oxime II dissolved in 0.5 mL DMF, to react with 1-[¹⁸F]fluoro-2-tosyloxyethane. The tittle compound is purified reverse phase semi-prep HPLC.

### EXAMPLE 3c

### Preparation of [¹⁸F]radicicol 9-(O-fluoroethyl-oxime), [¹⁸F]Ib (Ra=[¹⁸F]C₂H₄) from radicicol

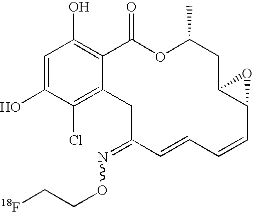

The title compound can be prepared by reacting [18F]fluoroethylamine, prepared according to literature procedures (Journal of Labelled Compounds & Radiopharmaceuticals (2002), 45(3), 217-229, Nuclear medicine and biology (1997 November), 24(8), 755-60), and radicicol according to literature procedure (Bioorganic & Medicinal Chemistry 2002, 19, 3445-3454), followed by reverse phase semi-prep HPLC purification.

### EXAMPLE 4

### Preparation of [¹¹C-OMe] radicicol 9-ylideneaminooxy-acetic acid methyl ester, [¹¹C]Ic (Rc=CH₂CO₂Me)

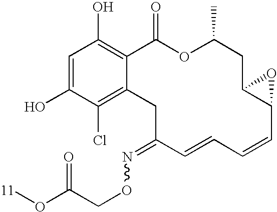

The title compound can be prepared by reacting [¹¹C]MeI with the sodium salt of the carboxylic acid V in dry DMF. [¹¹C]MeI is trapped at room into a 1 ml glass container loaded with reactants, when addition is completed the reaction mixture is heated. The title compound is then purified by reverse phase semi-prep HPLC.

### EXAMPLE 5

### Preparation of [O-tri(3H)-methyl] radicicol 9-ylideneaminooxy-acetic acid methyl ester, [³H]Ic (Rc=CH₂COMe)

The title compound can be prepared by reacting [³H]MeI with the sodium or potassium salt of the carboxylic acid V in dry DMF, followed by reverse phase semi-prep HPLC purification.

### EXAMPLE 6a

### Preparation of radicicol 9-ylideneaminooxy-N-(2-fluoro-ethyl)-acetamide, Id (Rd=CH₂CONHC₂H₄F)

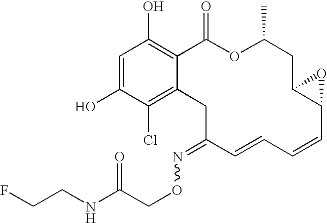

The title compound can be prepared according to literature procedure [J. Med. Chem. 2003, 46, 2534-2541] using respectively the hydrochloride salt of 2-fluoro-ethylamine, the hydrochloride salt of methylamine or ammonium hydrochloride.

Diastereoisomeric mixture of 2-[(9Z, 11E)-(4R,6R,8R)-16-Chloro-17,19-dihydroxy-4-methyl-2-oxo-3,7-dioxa-tricyclo[13.4.0.0*6,8*]nonadeca-1(19),9,11,15,17-pentaen-13-ylideneaminooxy]-N-(2-fluoro-ethyl)-acetamide, HPLC t_{R}: 4.3, (M+H)⁺=483, (M+H)⁻=482.

### EXAMPLE 6b

### Preparation of radicicol 9-ylideneaminooxy-N-methyl-acetamide, Id (Rd=CH₂CONHCH₃)

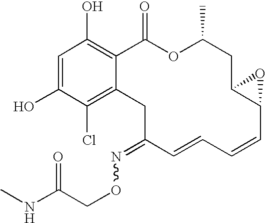

The title compound can be prepared according to literature procedure [J. Med. Chem. 2003, 46, 2534-2541] using respectively the hydrochloride salt of 2-fluoro-ethylamine, the hydrochloride salt of methylamine or ammonium hydrochloride.

Diastereoisomeric mixture of 2-[(9Z, 11 E)-(4R,6R,8R)-16-Chloro-17,19-dihydroxy-4-methyl-2-oxo-3,7-dioxa-tricyclo[13.4.0.0*6,8*]nonadeca-1 (19),9,11,15,17-pentaen-13-ylideneaminooxy]-N-methyl-acetamide, HPLC t_{R}: 4.1, (M+H)⁻=449.

### EXAMPLE 6c

### Preparation of radicicol 9-ylideneaminooxy-acetamide, Id (Rd=CH₂CONH₂)

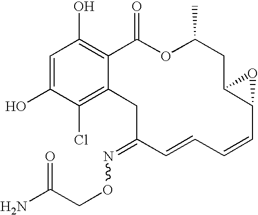

The title compound can be prepared according to literature procedure [J. Med. Chem. 2003, 46, 2534-2541] using respectively the hydrochloride salt of 2-fluoro-ethylamine, the hydrochloride salt of methylamine or ammonium hydrochloride.

Diastereoisomeric mixture of 2-[(9Z,11E)-(4R,6R,8R)-16-Chloro-17,19-dihydroxy-4-methyl-2-oxo-3,7-dioxa-tricyclo[13.4.0.0*6,8*]nonadeca-1 (19),9,11,15,17-pentaen-13-ylideneaminooxy]-acetamide, HPLC t_{R}: 3.9, (M+H)⁻=435.

### EXAMPLE 6d

### Preparation of radicicol 9-ylideneaminooxy-N-methyl-acetamide, Id (Rd=CH₂CONHCH₃)

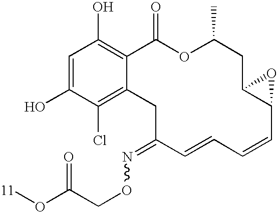

The title compound can be prepared by reacting [¹¹C]MeI with Example 5c in presence of a basein dry DMF. [¹¹C]MeI is trapped at room into a 1 ml glass container loaded with reactants, when addition is completed the reaction mixture is heated. The title compound is then purified by reverse phase semi-prep HPLC.

### EXAMPLE 6e

### Preparation of [¹⁸F] radicicol 9-ylideneaminooxy-N-(2-fluoro-ethyl)-acetamide, [18F]Id (x=F, n=2, 1)

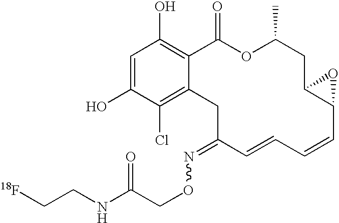

(Condensation of Radicicol 13-Ylideneaminooxy-Acetic Acid with 2-Fluoro-Ethylamine)

The first step consists of the preparation of 2-[18F]fluoroethylamine via cryptate mediated n.c.a. 18F-fluorination of N-Boc-2-(p-toluenesulfonyloxy)ethylamine in DMSO and subsequent deprotection as described in Journal of Labelled Compounds & Radiopharmaceuticals (2002), 45(3), 217-229. The title compound can be prepared by reacting the carboxylic acid V (n=1) with 2-[18F]fluoroethylamine followed by reverse phase semi-prep HPLC purification.

### EXAMPLE 7

### Ki/IC50 Determination (Binding Assay)

The inhibition of Hsp90 is measured using the procedure, with minor modifications, described in Schilb et al. Development and Implementation of a Highly Miniaturized Confocal 2D-FIDA-Based Analysis-Based High-Throughput Screening Assay to Search for Active Site Modulators of the Human Heat Shock Protein 90p, J of Biomolecular Screening, 2003 in press. The procedure is repeated for different concentrations of test compound selected to cover the range of 0% to 100% inhibition and the concentration at which 50% inhibition of Hsp90 occurs (IC₅₀) for each compound is determined from concentration-inhibition curves in a conventional manner.

The compounds of the Examples hereinbelow have IC₅₀ values of the order of 50-1000 nM or less in the above mentioned FIDA assay, specifically ≦1100 nM.

### EXAMPLE 8

### Pharmacokinetics and Organ Distribution

Tissue distribution of radioactivity is determined in normal and tumor-bearing male Fischer rats (200-250 g) after intravenous injection of the radiolabelled compound (1-100 MBq). The animals are allowed food and water ad libitum before the experiment. Following anesthesia radiopharmaceutical is injected into the rats via a tail vein catheter. Groups of four rats are sacrificed at various time points after injection of the dose. The animals are dissected, and selected tissues are weighed and counted along with dose standards in a Gamma Counter. The raw counts are decay-corrected, and the counts normalized as the percent of total injected dose per gram of tissue (% ID/g).

The tissue distribution of radioactivity is also determined in tumor-bearing rats following intravenous injection. The procedure is similar to that already described for normal rats. The same tissues are assayed as in normal rats with the addition of the tumor tissue, and the corresponding region of brain contralateral to the tumor is excised and used for comparison.

### EXAMPLE 9

### Ex Vivo Autoradiography

The regional distribution and uptake of the radiopharmaceuticals can be investigated using quantitative autoradiographic techniques. Radiopharmaceuticals are injected into the tail vein of the animal. Tissues are immediately removed and frozen in isopentane, which is cooled to −70° C. The frozen samples are cut into 20 μm sagittal sections using a cryostate, mounted on glass microscope slides and without any washing they are placed on a phosphor imager screen for 2 h. The imaging plate data is analysed by BAS800 II system (Fuji Film).

1. A compound of the formula I, 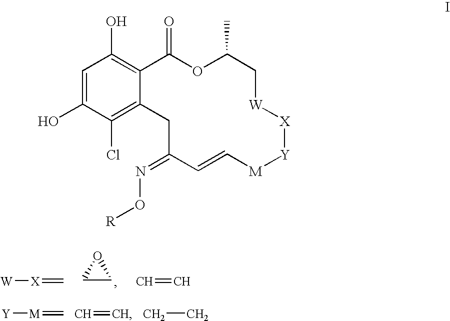
in which
R is R′, (CH₂)ₙCOOR′ or (CH₂)ₙCONHR′, wherein
R′ is ¹¹CH₃, [³H]₃C, [³H]₂HC, [³H]H₂C or (CH₂)ₙHal, wherein
Hal is ¹²³I, ¹²⁵I, ¹³¹I, I, ⁷⁵Br, ⁷⁶Br, ⁷⁷Br, ⁸²Br, Br, ¹⁸F or F, or
R′ is (CH₂)ₙ₋₁[³H]HCHal or (CH₂)ₙ₋₁[³H]₂CHal, wherein
Hal is I, Br or F, and
n is, each independently, 1, 2, 3 or 4,
in free form or in salt form.

2. A The compound according to claim 1 of the formula I, in which
R is R′, (CH₂)ₙCOOR′ or (CH₂)ₙCONHR′, wherein
R′ is ¹¹CH₃, [³H]₃C, [³H]₂HC, [³H]H₂C or (CH₂)ₙHal, wherein
Hal is ¹²³I, ¹²⁵I, ¹³¹I, I, ⁷⁵Br, ⁷⁶Br, ⁷⁷Br, ⁸²Br or ¹⁸F or
R′ is (CH₂)ₙ₋₁[³H]HCHal or (CH₂)ₙ₋₁[³H]₂CHal, wherein
Hal is I, Br or F, and
n is, each independently, 1, 2, 3 or 4.

3. A process for the preparation of the compound as defined in claim 1 of the formula I, in free form or in salt form, comprising the steps of
a) for the preparation of a compound of the formula Ia, 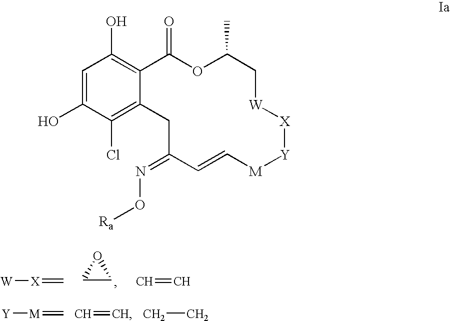
in which Rₐ is ¹¹CH₃, [³H]H₂C, [³H]₂HC or [³H]₃C, reacting a compound of the formula II 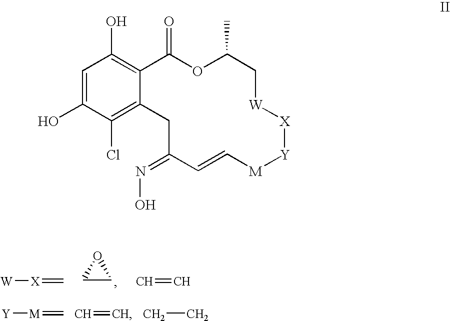
with ¹¹CH₃L, [³H]H₂CL, [³H]₂HCL or [³H]₃CL, wherein L is 1, OTs, OMs or OTf, in the presence of a base, or
b) for the preparation of a compound of the formula Ib, 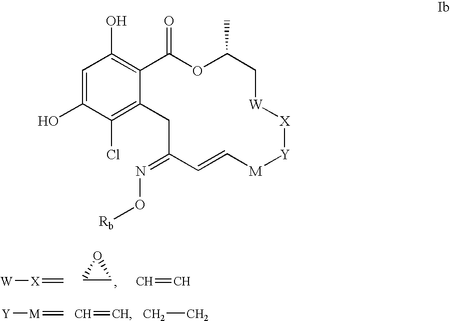
in which R_{b} is [¹⁸F](CH₂)ₙ, F(CH₂)ₙ, [¹²³I](CH₂)ₙ, [¹²⁵I](CH₂)ₙ, [¹³¹I](CH₂)ₙ, I(CH₂)ₙ, [⁷⁵Br](CH₂)ₙ, [⁷⁶Br](CH₂)ₙ, [⁷⁷Br](CH₂)ₙ, [⁸²Br](CH₂)ₙ, or Br(CH₂)ₙ, reacting a compound of the formula III, 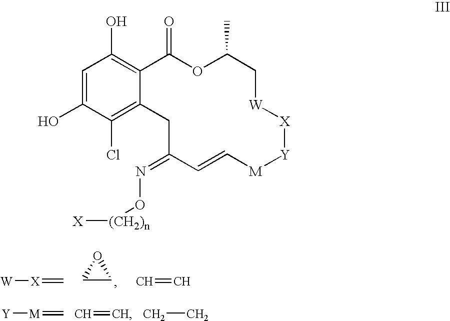
in which n is 1, 2, 3 or 4 and X [in the group ═N—O—(CH₂)ₙ—X] is OTs, OMs, OTf or 1, with [¹²³I]⁻, [¹²⁵I]⁻, [¹³¹I]⁻, I⁻, [⁷⁵Br]⁻, [⁷⁶Br]⁻, [⁷⁷Br]⁻, [⁸² Br]⁻, Br⁻, [¹⁸F]⁻ or F⁻
or reacting a compound of the formula II with a compound of the formula IV,
X—R_{b} IV
wherein X and R_{b} are as defined above in this variant,
or reacting a radicicol derivative, which carries an oxo group instead of the oxime function present in the compound of the formula I, with [¹⁸F](CH₂)ₙ—O—NH₂, [¹²³I](CH₂)ₙ—O—NH₂, [¹²⁵I](CH₂)ₙ—O—NH₂, [¹³¹I](CH₂)ₙ—O—NH₂, [⁷⁵Br](CH₂)ₙ—O—NH₂, [⁷⁶Br](CH₂)ₙ—O—NH₂, [⁷⁷Br](CH₂)ₙ—O—NH₂ or [⁸²Br](CH₂)ₙ—O—NH₂, n being in each case 1, 2, 3 or 4, preferably 2, 3 or 4, or
c) for the preparation of a compound of the formula Ic, 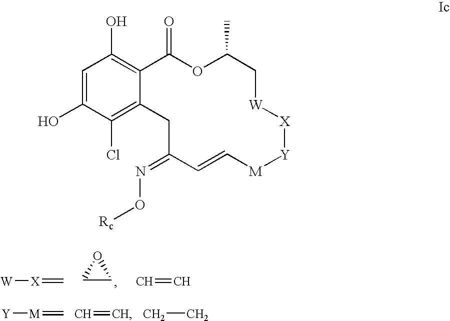
wherein R_{c} is (CH₂)ₙCO₂[¹¹C]H₃, (CH₂)ₙCO₂[³H]₃C, (CH₂)ₙCO₂[³H]₂HC or (CH₂)ₙCO₂[³H]H₂C, reacting a compound of the formula V, 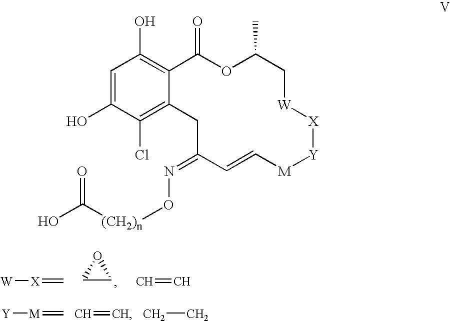
in which n is 1, 2, 3 or 4, with [¹¹C]H₃L, [³H]H₂CL, [³H]₂HCL or [³H]₃CL, wherein L is I, OTs, OMs or OTf, in the presence of a base, or
d) for the preparation of a compound of the formula Id, 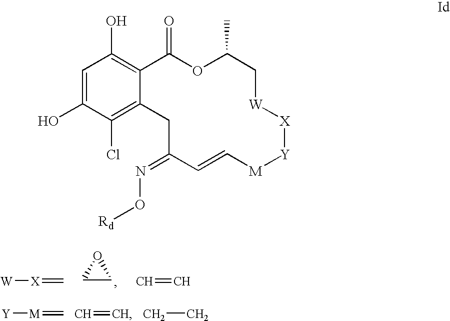
wherein R_{d} is [¹²³I](CH₂)ₙCONH(CH₂)ₙI, [¹²⁵I](CH₂)ₙCONH(CH₂)ₙI, [¹³¹I](CH₂)ₙCONH(CH₂)ₙI, (CH₂)ₙCONH(CH₂)ₙI, [⁷⁵Br](CH₂)ₙCONH(CH₂)ₙBr, [⁷⁶Br](CH₂)ₙCONH(CH₂)ₙBr, [⁷⁷Br](CH₂)ₙCONH(CH₂)ₙBr, [⁸²Br](CH₂)ₙCONH(CH₂)ₙBr, (CH₂)ₙCONH(CH₂)ₙBr, [¹⁸F](CH₂)ₙCONH(CH₂)ₙF, or (CH₂)ₙCONH(CH₂)ₙF, n being in each case, independently, 1, 2, 3 or 4, reacting a compound of the formula VI, 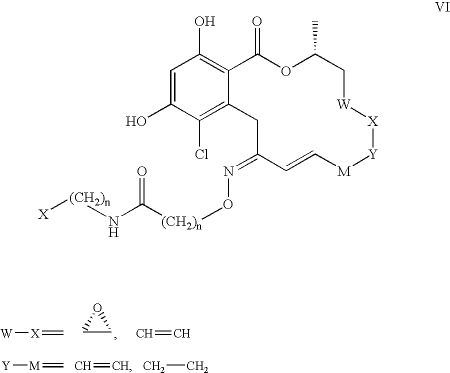
wherein n is in each case, independently, 1, 2, 3 or 4 and the terminal X is OTs, OMs, OTf or 1, with [¹²³I]⁻, [¹²⁵I]⁻, [¹³¹I]⁻, I⁻, [⁷⁵Br]⁻, [⁷⁶Br]⁻, [⁷⁷Br]⁻, [⁸² Br]⁻, Br⁻, [¹⁸F]⁻ or F⁻
or reacting a compound of the formula V, in which n is 1, 2, 3 or 4, with [¹⁸F](CH₂)ₙNH₂, F(CH₂)ₙNH₂, [¹²³I](CH₂)ₙNH₂, [¹²⁵I](CH₂)ₙNH₂, [¹³¹I](CH₂)ₙNH₂, I(CH₂)ₙNH₂, [⁷⁵Br](CH₂)ₙNH₂, [⁷⁶Br](CH₂)ₙNH₂, [⁷⁷Br](CH₂)ₙNH₂, [⁸²Br](CH₂)ₙNH₂, Br(CH₂)ₙNH₂, F[³H]₃C(CH₂)ₙ₋₁NH₂, F[³H]₂HC(CH₂)ₙ₋₁NH₂, F[³H]H₂C(CH₂)ₙ₋₁NH₂, Br[³H]₃C(CH₂)ₙ₋₁NH₂, Br[³H]₂HC(CH₂)ₙ₋₁NH₂, Br[³H]H₂C(CH₂)ₙ₋₁NH₂, I[³H]₃C(CH₂)ₙ₋₁NH₂, I[³H]₂HC(CH₂)ₙ₋₁NH₂ or I[³H]H₂C(CH₂)ₙ₋₁NH₂, n being in each case 2, 3 or 4, using standard carboxylic activation methods,
in each case optionally followed by cleavage of protecting groups optionally present, and in each case of recovering the so obtainable compound of the formula I in free, preferably free acid, form or in the form of a, preferably phenolic, salt.

4. A radiotracer/marker, comprising:
the compound of the formula I as defined in claim 1.

5. (canceled)

6. A labeling composition for labeling tumors and other tissues involving overexpression or activation of Hsp90 in vivo or in vitro, comprising:
the compound of the formula I as defined in claim 1.

7. A method for labeling tumors and other tissues involving overexpression or activation of Hsp90 in vivo or in vitro, comprising:
contacting the tumor tissue or the other tissues with the compound of the formula I as defined in claim 1.
